(19) United States
(12) Patent Application Publication
(10) Pub. No.: US 2026/0183517 A1
(43) Pub. Date: Jul. 2, 2026

(54) SECONDARY SECUREMENT DEVICE FOR VASCULAR ACCESS DEVICE

(71) Applicant: BECTON, DICKINSON AND COMPANY, Franklin Lakes, NJ (US)

(72) Inventors: Rahul Malviya, Salmon Arm (CA); Divik Bhargava, Meerut (IN); Atharva Shetye, Kolhapur (IN); Srimannarayana Myla, Pune (IN)

(21) Appl. No.: 19/130,993

(22) PCT Filed: Jan. 11, 2023
(86) PCT No.: PCT/US2023/010562

§ 371 Date: May 19, 2025

(51) Int. Cl. A61M 25/02

(52) U.S. Cl. CPC A61M 25/02; A61M 2209/088; A61M 2210/04

(57) ABSTRACT

A secondary securement device for a vascular access device comprising tubing, the secondary securement device including a base, a first securing member, and a second securing member. The first securing member including a first end connected to the base and a second end positioned opposite from the first end, with the second end moveable relative to the base. The second securing member including a first end connected to the base and a second end positioned opposite from the first end, with the second end moveable relative to the base. The first securing member is configured to secure the tubing of the vascular access device at a first location, and the second securing member is configured to secure the tubing of the vascular access device at a second location spaced from the first location.

### BACKGROUND OF THE INVENTION

### Field of the Invention

The present disclosure relates to a secondary securement device for a vascular access device.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal. Common types of VADs are an over-the-needle peripheral intravenous catheter (PIVC) or central venous catheter (CVC). A variety of solutions exist for securing VADs and maintaining the insertion site; however, clinicians are often left with limited solutions to manage the portions of the lumen that extend outside of the body. These loose extensions can create opportunities for the lumens to be pulled, potentially compromising the catheter position or insertion site integrity. Tape and textile dressings are sometimes utilized, but can be cumbersome when the line needs to be accessed. In addition, while a line is being accessed, an un-capped line is at risk for touch contamination as the clinician often needs to perform multiple steps.

### SUMMARY OF THE INVENTION

In one aspect or embodiment, a secondary securement device for a vascular access device having tubing, includes a base including a first end, a second end positioned opposite the first end, a first side, a second side positioned opposite the first side, a top surface, and a bottom surface positioned opposite the top surface, with the bottom surface configured to be in contact with a patient's skin surface, a first securing member including a first end connected to the base and a second end positioned opposite from the first end, with the second end moveable relative to the base, and a second securing member including a first end connected to the base and a second end positioned opposite from the first end, with the second end moveable relative to the base. The first securing member is configured to secure the tubing of the vascular access device at a first location and the second securing member is configured to secure the tubing of the vascular access device at a second location spaced from the first location.

The first ends of the first and second securing members are connected to the body at a position intermediate the first and second ends of the base, where the first securing member extends toward the first end of the base and the second securing member extends toward the second end of the base. The secondary securement device may further includes a first retainer connected to the base and a second retainer spaced from the first retainer and connected to the base, where a portion of the first securing member is positioned between the first retainer and the base and a portion of the second securing member is positioned between the second retainer and the base. The first and second retainers may each be a slit defined by the base. The bottom surface of the base may include an adhesive surface and a liner. The first and second securing members may each be flexible fabric tape. The first and second securing members may each include an adhesive surface. The base may be flexible.

In a further aspect or embodiment, a method of using the secondary securement device of any of the aspects or embodiments discussed above may include: positioning the tubing of the vascular access device between the first securing member and the base at the first location; and positioning the tubing of the vascular access device between the second securing member and the base at the second location.

The method may further include: pulling the second end of the first securing member to tighten the first securing member onto the tubing; and pulling the second end of the second securing member to tighten the second securing member onto the tubing. The tubing of the vascular access device may have a J shape. The method may further include removing a liner from the bottom surface of the base; and adhering the base to a skin surface of a patient.

### BRIEF DESCRIPTION OF THE DRAWINGS

FIG. 1 is a perspective view of a secondary securement device for a vascular access device according to one aspect or embodiment of the present application;

FIG. 2 is a perspective view of the secondary securement device of FIG. 1;

FIG. 3 is a perspective view of the secondary securement device of FIG. 1, showing removal of a liner;

FIG. 4 is a perspective view of the secondary securement device of FIG. 1, showing the secondary securement device in use;

FIG. 5 is a perspective view of the secondary securement device of FIG. 1, showing the secondary securement device being secured to tubing of a vascular access device;

FIG. 6 is a perspective view of the secondary securement device of FIG. 1, showing the secondary securement device being tightened;

FIG. 7 is a perspective view of the secondary securement device of FIG. 1, showing the secondary securement device secured to tubing of a vascular access device; and

FIG. 8 is a perspective view of the secondary securement device of FIG. 1, showing the secondary securement device in use with multiple tubing.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

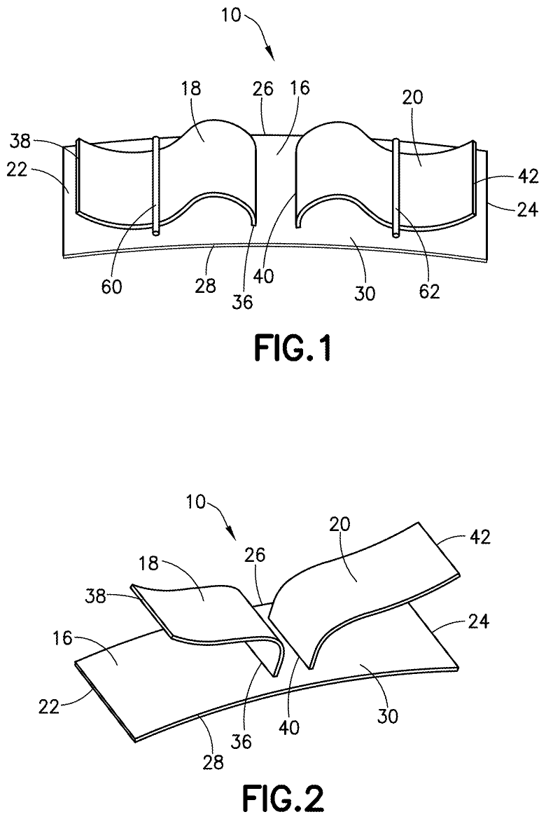

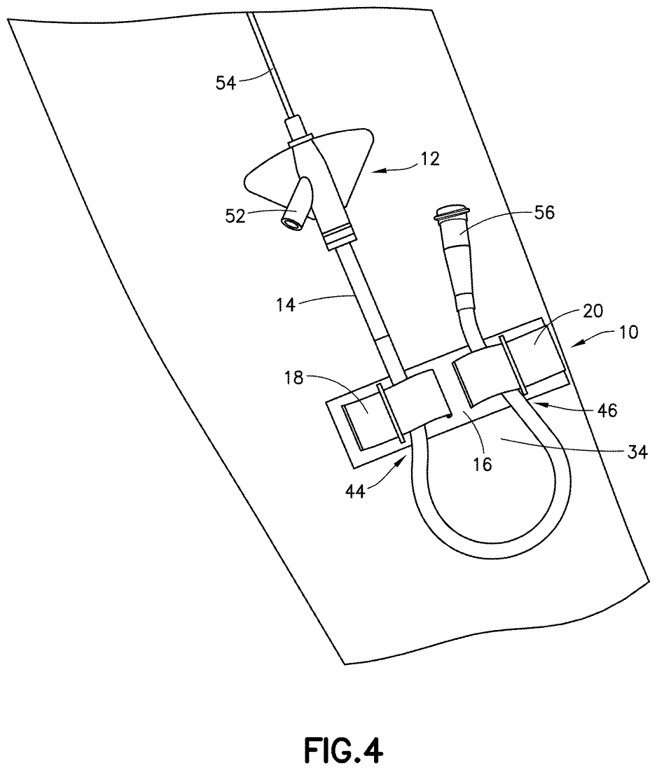

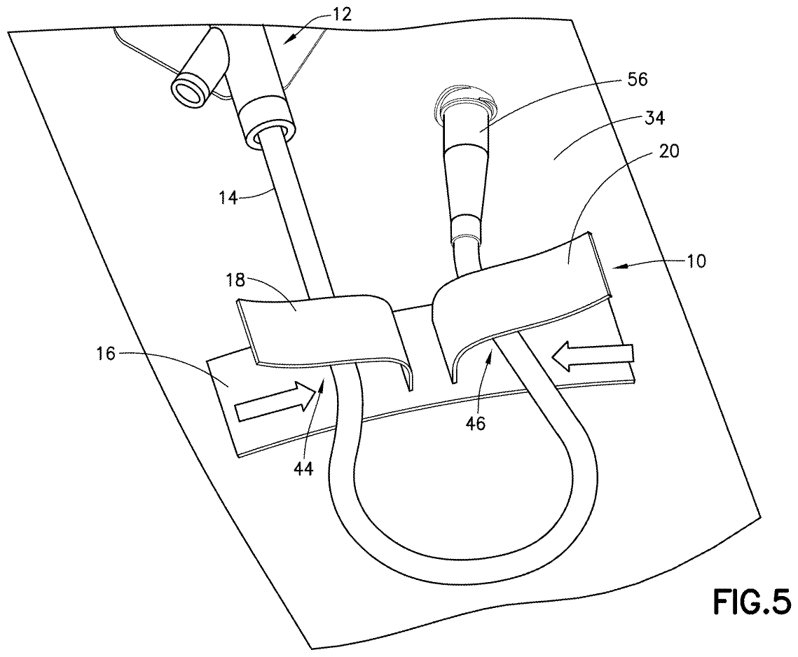

Referring to FIGS. 1-8, in one aspect or embodiment, a secondary securement device 10 for a vascular access device 12 including tubing 14 is shown. The secondary securement device 10 includes a base 16, a first securing member 18, and a second securing member 20. The base 16 includes a first end 22, a second end 24 positioned opposite the first end 22, a first side 26, a second side 28 positioned opposite the first side 26, a top surface 30, and a bottom surface 32 positioned opposite the top surface 30, with the bottom surface 32 configured to be in contact with a patient's skin surface 34. The first securing member 18 includes a first end 36 connected to the base 16 and a second end 38 positioned opposite from the first end 36, with the second end 38 moveable relative to the base 16. The second securing member 20 includes a first end 40 connected to the base 16 and a second end 42 positioned opposite from the first end 40, with the second end 42 moveable relative to the base 16. The first securing member 18 is configured to secure the tubing 14 of the vascular access device 12 at a first location 44 and the second securing member 20 is configured to secure the tubing 14 of the vascular access device 12 at a second location 46 spaced from the first location 44.

As discussed in additional detail below, the secondary securement device 10 is configured to hold and grip the tubing 14 of the vascular access device 12 to prevent accidental pull forces from transmitting to the vascular access device 12 and placed catheter. The vascular access device 12 may be a peripheral intravenous catheter or central venous catheter, although the secondary securement device 10 may be utilized in connection with other medical devices. In some aspects or embodiments, the vascular access device 12 includes a catheter adapter 52, a catheter 54, the tubing 14, and a connector 56 positioned at a proximal end of the tubing 14.

Referring to FIGS. 1 and 2, the first ends 36, 40 of the first and second securing members 18, 20 are connected to the base 16 at a position intermediate the first and second ends 22, 24 of the base 16, with the first securing member 18 extending toward the first end 22 of the base 16 and the second securing member 20 extending toward the second end 24 of the base 16. In other words, the first and second securing members 18, 20 are configured to extend away from each other. The first ends 36, 40 of the first and second securing members 18, 20 are spaced from each other. As shown in FIG. 1, a first retainer 60 is connected to the base 16 and a second retainer 62 is spaced from the first retainer 60 and connected to the base 16. A portion of the first securing member 18 is positioned between the first retainer 60 and the base 16 and a portion of the second securing member 20 is positioned between the second retainer 62 and the base 16. The first and second retainers 60, 62 may each be formed by a slit or slits defined by the base 16, although the first and second retainers 60, 62 may be a protrusion and/or separate structure connected to the base 16. In some aspects or embodiments, the first and second securing members 18, 20 are pre-assembled with the second ends 38, 42 of the first and second securing members 18, 20 threaded or positioned through the respective first and second retainer 60, 62. The first and second securing members 18, 20 may be formed from flexible fabric tape. The first and second securing members 18, 20 may have an adhesive surface and/or be formed from a material having gripping properties. The base 16 may be flexible and configured to conform to the patient skin surface 34.

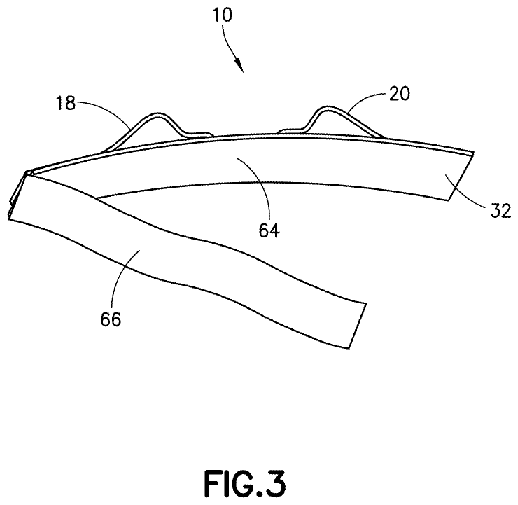

Referring to FIG. 3, in some aspects or embodiments, the bottom surface 32 of the base 16 includes an adhesive surface 64 and a liner 66. The liner 66 is configured to be peeled or removed from the adhesive surface 64 with the bottom surface 32 configured to be secured to the patient's skin 34 via the adhesive surface 64. The base 16 of the secondary securement device 10, however, may be secured to the patient's skin surface 34 via tape, dressing, or other suitable arrangements.

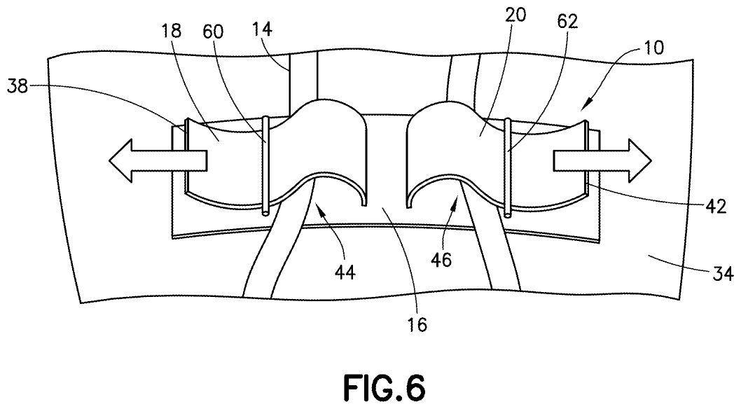

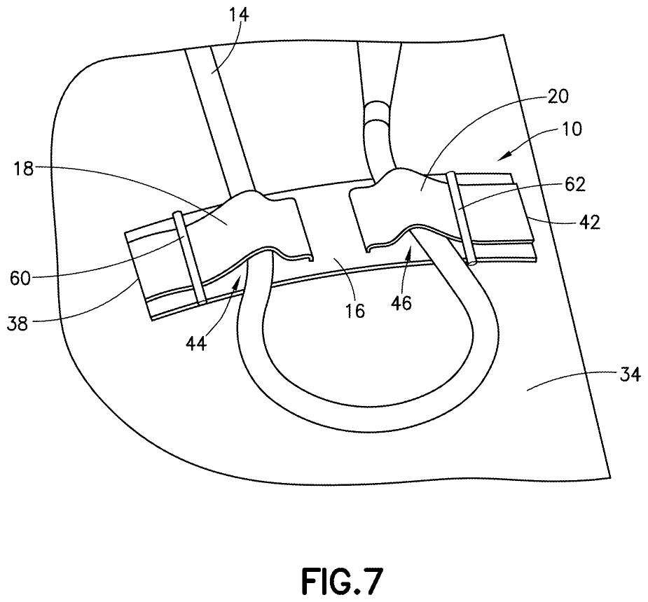

Referring to FIGS. 5-7, in one aspect or embodiment, a method of using the secondary securing device 10 includes: positioning the tubing 14 of the vascular access device 12 between the first securing member 18 and the base 16 at the first location 44; and positioning the tubing 14 of the vascular access device 12 between the second securing member 20 and the base 16 at the second location 46. As shown in FIG. 6, the method may further include: pulling the second end 38 of the first securing member 18 to tighten the first securing member 18 onto the tubing 14; and pulling the second end 42 of the second securing member 20 to tighten the second securing member 20 onto the tubing 14. As shown in FIG. 5, the tubing 14 of the vascular access device 12 has a J-shape or J-loop. The method may also include: removing the liner 66 from the bottom surface 32 of the base 16; and adhering the base 16 to the skin surface 34 of the patient. After pulling the first and second securing members 18, 20 to tighten the first and second securing members 18, 20 onto the tubing 14, the second ends 38, 42 of the securing members 18, 20 may be secured to the base 16 or the patient's skin surface 34. The first and second retainers 60, 62 are configured to provide leverage to ensure the first and second securing members 18, 20 tighten against the tubing 14 when pulled tight.

Although two securing members 18, 20 are shown, two or more securing members 18, 20 may be provided for securing multiple lines and/or connectors. The first and second securing members 18, 20 are configured to secure tubing having a variety of diameters.

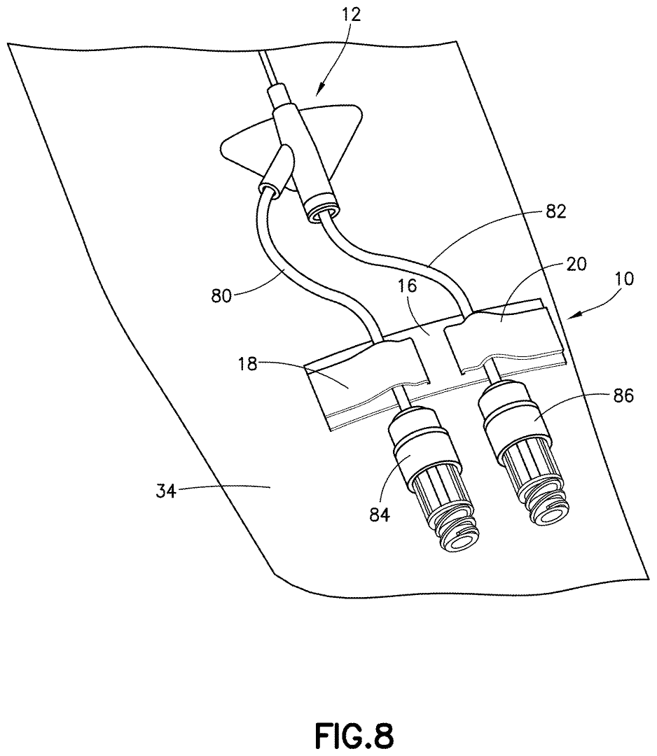

Referring to FIG. 8, in one aspect or embodiment, the vascular access device 10 includes a first line 80 and a second line 82 extending from the catheter adapter 52 or from the connector 56. The first line 80 includes a first port 84 and the second line 82 includes a second port 86. The first and second ports 84, 86 may each include a needle-free connector. The first securing member 18 is configured to secure the first line 80 and the second securing member 20 is configured to secure the second line 82.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A secondary securement device for a vascular access device comprising tubing, the secondary securement device comprising:
a base including a first end, a second end positioned opposite the first end, a first side, a second side positioned opposite the first side, a top surface, and a bottom surface positioned opposite the top surface, the bottom surface configured to be in contact with a patient's skin surface;
a first securing member including a first end connected to the base and a second end positioned opposite from the first end, the second end moveable relative to the base;
a second securing member including a first end connected to the base and a second end positioned opposite from the first end, the second end moveable relative to the base, wherein the first securing member is configured to secure the tubing of the vascular access device at a first location and the second securing member is configured to secure the tubing of the vascular access device at a second location spaced from the first location.

2. The secondary securement device of claim 1, wherein the first ends of the first and second securing members are connected to the body at a position intermediate the first and second ends of the base, and wherein the first securing member extends toward the first end of the base and the second securing member extends toward the second end of the base.

3. The secondary securement device of claim 1, further comprising a first retainer connected to the base and a second retainer spaced from the first retainer and connected to the base, wherein a portion of the first securing member is positioned between the first retainer and the base and a portion of the second securing member is positioned between the second retainer and the base.

4. The secondary securement device of claim 3, wherein the first and second retainers each comprise a slit defined by the base.

5. The secondary securement device of claim 1, wherein the bottom surface of the base comprises an adhesive surface and a liner.

6. The secondary securement device of claim 1 wherein the first and second securing members each comprise flexible fabric tape.

7. The secondary securement device of claim 1, wherein the first and second securing members each comprise an adhesive surface.

8. The secondary securement device of claim 1, wherein the base is flexible.

9. A method of using the secondary securement device of claim 1, the method comprising:
positioning the tubing of the vascular access device between the first securing member and the base at the first location; and
positioning the tubing of the vascular access device between the second securing member and the base at the second location.

10. The method of claim 9, wherein the secondary securement device comprises a first retainer connected to the base and a second retainer spaced from the first retainer and connected to the base, wherein a portion of the first securing member is positioned between the first retainer and the base and a portion of the second securing member is positioned between the second retainer and the base, the method further comprising:
pulling the second end of the first securing member to tighten the first securing member onto the tubing; and
pulling the second end of the second securing member to tighten the second securing member onto the tubing.

11. The method of claim 9, wherein the tubing of the vascular access device has a J-shape.

12. The method of claim 9, further comprising:
removing a liner from the bottom surface of the base; and
adhering the base to a skin surface of a patient.
